(19) United States
(12) Patent Application Publication
(10) Pub. No.: US 2007/0213682 A1
(43) Pub. Date: Sep. 13, 2007

(54) Penetration device, kit, and method

(76) Inventors: Hans-Peter Haar, Wiesloch (DE); Hans List, Hesseneck-Kailbach (DE); Irio Guiseppe Calasso, Arth (CH)

Correspondence Address: THE LAW OFFICE OF JILL L. WOODBURN, L.L.C.;JILL L. WOODBURN, 128 SHORE DR., OGDEN DUNES, IN, 46368, US

(21) Appl. No.: 11/373,902

(22) Filed: Mar. 13, 2006

(51) Int. Cl. A61M 31/00

(52) U.S. Cl. 604/500

(57) ABSTRACT

The present invention provides a method and device for extracting body fluid from a body part. The method provides a penetration device including a penetration element formed to pierce the body part and an actuator formed to propel forward movement of the penetration element. The penetration element is moved toward the body part at a very high penetration speed to form an impression in the body part having a depth less than about 0.3 mm and the body part is pierced with the penetration element. The actuator of the penetration device is formed to propel forward movement of the penetration element at a penetration speed of at least about 15 m/sec.

### TECHNICAL FIELD

The invention concerns a penetration device and method for the extraction of body fluid with a penetration element that can prick the skin of a body part and an actuator formed to drive penetration movement of the penetration element.

### BACKGROUND

Self-monitoring tests requiring a small volume of blood are well known. For example, test kits for measurement of blood sugar levels are utilized by diabetics. These kits require that a drop of blood be placed on a test strip that cooperates with a hand-held (or portable) measurement apparatus, which in turn displays the glucose concentration in the blood sample. To obtain the drop of blood, the user is typically supplied with a lancet device, which makes a skin prick, typically in the user's finger.

The condition of the skin has had a significant effect on the penetration of the lancet into it. Because of the ability of the skin to stretch, a significant variation in a depth of penetration of the lancet between different lancing episodes was possible. In prior technology, lancets were launched at speeds of about 1-5 m/sec. Using that technology, it is understood that a variation in skin impression and tissue movement prior to penetration was in the range of about 0.5 mm up to about 1 mm.

The observed variation in the penetration depth among different lancing episodes for a non-stabilized skin surface is attributed to the fact that, below a given penetration force level, there is a first linerly elastic range of the skin movement followed by an inelastic response until finally the lancet pierces with further stretching of the skin at the lancing site. Such a difference in distance between the first touching of the skin and the entry of the skin by the lancet depending on imperfections of the skin can lead to considerable variations in the actual penetration depth.

A skin stabilizer was therefore proposed as a remedy, which is pressed on the skin prior to the lancet penetrating the skin. See, U.S. Pat. No. 6,306,152 B1, The sequence, however, of movement must be controlled and possibly the blood is displaced by the pressed skin stabilizer.

### SUMMARY

The present invention provides a puncturing device and method formed to reach a definite penetration depth for improvement of the extraction of body fluid such as blood.

The invention is based on the idea that avoidance of the extensive skin movement during penetration may be achieved by taking advantage of the inertia of the concerned tissue parts. Accordingly, the instant invention uses an actuator in the form of a high-speed drive to bring the penetration element to a very high penetration speed that a penetration depth variation caused by skin impression or compression is less than about 0.3 mm, more particularly less than about 0.2 mm. Thus, in the instant invention the penetration depth no longer significantly depends on the elasticity or imperfections of the skin, but can be specifically and individually defined. No special measures are imposed on the penetration element for this, which is especially favorable for disposable units. Due to reduced penetration depth inaccuracy, a person can adjust the penetration device better to be reproducible in order to achieve an optimum penetration depth in the sense of a successful fluid extraction and reduction of the penetration pain. The skin is less traumatized by not penetrating unnecessarily deep, such that, due to increased convenience, prescribed test routines are more likely maintained, which is important for blood sugar tests in diabetes care.

An embodiment of the invention includes a method for extracting body fluid from a body part, the method comprising providing a penetration device including a penetration element formed to pierce the body part and an actuator formed to propel forward movement of the penetration element, moving the penetration element toward the body part at a very high penetration speed to form an impression in the body part having a depth less than about 0.3 mm, and piercing the body part with the penetration element.

Another embodiment provides a penetration device for the extraction of a body fluid. The device comprises a penetration element formed to pierce a skin of a body part, and actuating means for propelling a penetration movement of the penetration element, wherein the actuating means has a high-speed drive formed to bring the penetration element to such a high penetration speed that a penetration depth variation caused by skin impression is less than about 0.3 mm.

Another embodiment provides a kit for the extraction of body fluid from a body part. The kit comprises a pressing ring, a penetration element sized for extension into the pressing ring and formed to pierce the body part, a first actuator formed to propel forward movement of the penetration element at a penetration speed of at least about 15 m/sec, and a second actuator formed to propel a retraction of the penetration element from the body part.

It is appreciated that in at least one embodiment of the present invention it is the actuator that provides the penetration element with a penetration speed of at least about 15 m/sec. In another embodiment of the invention, the actuator provides the penetration element with a penetration speed of about 20 m/sec to about 30 m/sec, in order to reduce significantly the penetration depth variation caused by skin impression.

Further, for the collection of the body fluid, an embodiment provides that a retraction speed for the retraction of the penetration element out of the skin amounts to a no more than about 10% of the penetration speed.

In order to simplify the acceleration and its braking, an embodiment provides that the mass of the penetrative part of the penetration element penetrating into the body part is less than about 25 mg.

An embodiment of the invention provides that the actuator comprise separate drives for the forward movement and the retracting movement of the penetration element. With that, the different speeds in the forward propulsion phase and retracting phrase can be controlled.

An embodiment of the invention provides that the actuator can create an electromagnetic impulse for the initiation of the forward movement of the penetration element. Another embodiment provides for that the actuator create an excess-pressure impulse triggering the forward movement of the penetration element, such as via a pneumatic or hydraulic cylinder or a pyrotechnic propelling charge.

In order to provide high energy storage density, an embodiment provides that the actuator comprises a preloaded spring element, such as a pre-compressed silicone rubber spring, for the forward movement of the penetration element.

Another embodiment provides that the actuator comprises a driving body, which can be accelerated and decelerated, separately from the penetration element, such as a flywheel or a plunger, for the transfer of a mechanical impulse to the penetration element.

In order to create a definite framework, an embodiment provides that the penetration element is supported in a carrier or housing and is linearly moveable along an acceleration distance. For limiting the forward movement, an embodiment provides that the housing has a stop for the penetration element.

A further embodiment proves that the actuator engages the carrier for the retracting movement of the penetration element, whereby the carrier forms a driver for the penetration element.

In an embodiment, the penetration element is formed by a lancet, a microsampler (a penetration unit with reception area for the sample) or an integrated system, with which not only the extraction of blood but also the analysis is carried out, without additional steps on the part of the user.

As regarding the method, an embodiment provides that the penetration speed of the penetration element is selected to be very high that a penetration depth variation caused by skin impression is significantly reduced, such that, the skin surface is compressed by less than about 0.3 mm. In an embodiment, the skin surface is compressed by less than about 0.2 mm prior to penetration of the penetration element. For this, the penetration speed of at least about 15 m/sec is selected.

A further embodiment provides that the penetration element is driven in the forward phase of the penetration movement in a non-controlled manner such that the penetration element is provided with a kinetic energy, which is at minimum an order of magnitude greater than the energy required for penetration into the skin, and the surplus kinetic energy of the penetration element at the end of the forward-directed penetration movement is transferred to the carrier as thrust.

Other features and embodiments of the present invention will become apparent from the following detailed description. It should be understood, however, that the detailed description and the specific examples, while indicating embodiments of the invention, are given by way of illustration only, since various changes and modifications within the spirit and scope of the invention will become apparent to those skilled in the art from this detailed description.

### BRIEF DESCRIPTION OF THE DRAWINGS

Below, the invention is explained in detail based on the embodiments represented schematically in the drawings. The following are shown respectively in the longitudinal cross section:

FIG. 1 shows a penetration device with a high speed drive for the penetration element in different phases of the penetration movement;

FIG. 2 shows an embodiment with a linear spring for the acceleration of the penetration element;

FIG. 3 shows an embodiment with fluidic drive transmission;

FIG. 4 shows an embodiment with flywheel drive for the penetration element;

FIG. 5 shows an embodiment for the electrodynamic acceleration of the penetration element; and

FIG. 6 shows an embodiment having a pyrotechnically working form.

### DETAILED DESCRIPTION

The penetration or lancing devices represented in the drawings comprise a penetration or lancing element 14 for pricking into the skin 10 of a body part 12 and an actuator 16 that is formed to propel forward movement of the penetration element 14 to a sufficiently high penetration speed in order to reduce penetration depth variation formed by skin deformation. Non-limiting examples of suitable uses of a penetration device 20 include blood extraction systems, stand-alone pricking apparatuses, micro-samplers and integrated penetration and analysis systems for blood sugar measurements for patient self-monitoring.

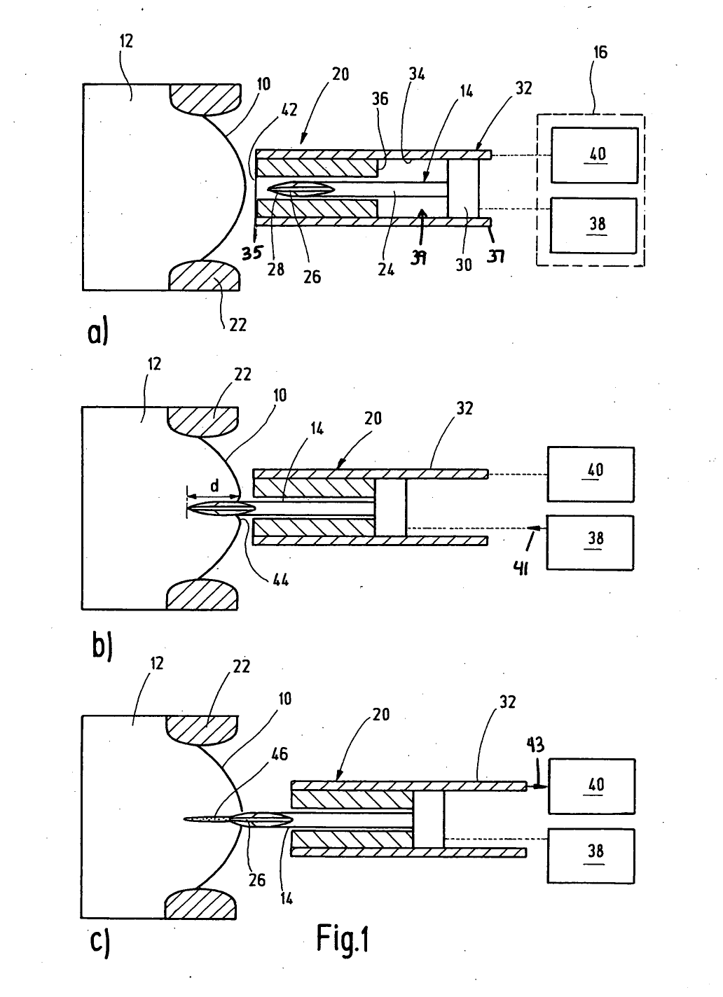

According to FIG. 1, a body part 12, in this instance a finger, is pressed against a pressing ring 22 of a portable instrument, such that the finger pad bulges inside the ring 22 to allow the penetration of the penetration element 14. It is appreciated that while the element 14 is sized for extension into an opening of the ring 22, the extent of any such extension depends upon the height that the body part has bulged upwardly through the opening. For example, when whole blood is sampled during the retraction movement of the penetration element 14, it is favourable that the skin/tissue area of e.g. the finger is compressed by the ring 22.

A diameter of the opening of the ring 22 is in the order of about 3 to about 8 mm. It is appreciated, that the diameter of the opening may vary depending upon the size of the body part e.g. the finger of e.g. a child or adult. It is further appreciated that the structure of the ring may be formed of a solid plastic or metal or may be flexible and formed of, for example a flexible plastic or rubber material. While the ring 22 may be a portion of a portable instrument, an additional non-limiting example of a compression unit suitable for use with the penetration element is found in U.S. Pat. No. 6.679,852 the disclosure of which is hereby incorporated by reference.

It is appreciated that while a finger is illustrated and described, than any number of body parts, non-limiting examples of which include hands, arms, legs, feet, toes, head, neck, stomach, chest, back, and buttocks, are within the scope of the present invention. The penetration speed of the drive of the penetration element 14 is selected to be so high that a concave impression 44 (FIG. 1b) of the skin 10 at the penetration site is significantly reduced during the penetration process. For example, the penetration element 14 is driven with a very high speed of at least about 15 m/sec. In an embodiment, the penetration speed when piercing the body part is about 20 m/sec to about 30 m/sec. In an embodiment, the penetration speed is about 30 m/sec.

The penetration element 14 shown in FIGS. 1(a-c) is positioned within a channel 39 of a housing or carrier 32. The penetration element 14 has an elongated shaft 24, at whose distal end a penetration tip 28 is grinded and a transversal collection slit 26 is provided. In an embodiment, the shaft 24 and the tip 28 of the penetration element 14 have a combined mass that is less than about 25 mg. The proximal end of the penetration element 14 is coupled to the drive through a coupling member 30. A non-limiting example of a suitable penetration element 14 is a lancet.

Penetration element 14 is illustrated and described as one example of a suitable lancet. As used herein, the term “lancet” refers to an elongated object with a sharp point for inserting into the skin to induce bleeding., it is understood that generally lancets may be needle-like with a round cross-section, or it may have cutting edge(s) along its elongated body for a cutting action to effect a less traumatic penetration into the skin in accordance with this disclosure. It is further appreciated in accordance with the present invention that the penetration element 14 may be a microsampler, or an integrated penetration and analyzing system. When the term “prick” and “pierce” is used herein, unless specified otherwise, it is to be understood that any of such penetration elements may be used.

The housing 32 has opposite distal and proximal ends 35, 37 and the channel 39 extends between the ends 35, 37. The penetration element 14 is capable of linearly traversing a pre-defined acceleration distance 34 in channel 39 of the housing 32. A stop 36 is provided in the housing 32 at the distal end of the acceleration distance 34 for the limitation of the forward movement, in the direction shown by arrow 41 in FIG. 1b of the penetration element 14. In an embodiment, longitudinal oscillations of the penetration element 14 are suppressed by suitable measures, such as a frictional contact (not shown) provided at the stop 36, for the avoidance of irritations.

In order to carry out the forward and retraction movements (41, 43) of the penetration element 14 with the desired speed profile, the actuator 16 contains a high speed drive 38 for the fast penetration phase and a second separate drive 40 for the slower retracting phase of the penetration movements. The drive 38 acts directly on the coupling member 30 of the penetration element 14, while the drive 40 for the retracting phase attaches to the housing 32.

The housing 32 can be closed at its distal end 35 with a penetrable foil 42 for sterile protection. The coupling member 30 can also provide a sealing function on the proximal end 37, so that the penetration tip 28 within the housing 32 remains sterile and protected from environmental influences. The housing 32 can also be a portion of a magazine (not shown), a non-limiting example of which includes a cylindrical magazine in order to provide a number of penetration elements 14 for successive penetrations. Before starting the penetration action, the penetration device 20 is preset to a desired penetration depth distance in the finger/ring-configuration, as is shown in FIG. 1a. It is appreciated that each individual should be able to precisely adjust the penetration depth to a value between about 0.5 mm and 3 mm to account for any boundary condition, for example even for different consistency of the skin in winter and summer.

In operation, the penetration element 14 pierces the body part to an extent, which permits the extraction of body fluid. When body fluid is extracted, the penetration depth distance following the piercing of the body part can be controlled by limiting the length the penetration element 14 protrudes from the distal end 35 of the housing 32. In the finger, the blood capillaries will end in the region where interstitial fluid is present, whereas the circumstances in the abdominal wall are different. In general, the dermis has a thickness of about 0.5 mm, and then each individual should be able to precisely adjust the penetration depth to a value between about 0.5 mm and 3 mm to account for any boundary condition, for example even for different consistency of the skin in winter and summer.

As shown in FIG. 1b, the drive 38 is activated for a quick forward penetration movement in direction 41. Due to the high penetration speed of the element 14 when piercing the body part being at least about 15 m/sec, the skin surface 10 in contact with the penetration tip 28 is compressed insignificantly, see impression 44, until the rupture limit is reached, i.e. the counter force to the cutting force of the penetration tip 28 effected by the tissue elasticity is exceeded. In this way, a pricking channel is created within the body part 12 with definite penetration depth (d). This pricking channel arises through the stop-limited forward movement 41 of the penetration element 14, while the variable skin impression 44 of the body part 12, which depends on the type and condition of the skin 10, is small.

The drive 38 brings the penetration element 14 to such a high penetration speed that the penetration depth variation caused by skin impression 44 is less than about 0.3 mm. In an embodiment, the penetration depth variation is less than about 0.2 mm. At the same time, the transverse oscillatory movements of the longitudinally guided penetration shaft 24 are avoided by the fast movement so that the penetration pain felt in the body part 12 is reduced.

A non-limiting estimate gives that for a penetration force of 0.3 N and a concerned tissue area of the assumed cube-shaped expansion of 1.5 mm as well as a penetration speed of 20 m/sec, the skin 10 is compressed by less than 0.1 mm per 1 mm penetration depth. The penetration depth variation caused by that lies therefore in the range of only 10%, so that a person intra-individually can adjust better to achieve an optimum with respect to the required penetration depth.

In order to reduce the equipment expense, it is appropriate if the penetration element 14 is driven in a non-controlled manner in the forward penetration phase of the penetration movement by the drive 38. This means that at least a significant part of the kinetic energy is transferred to the penetration element 14 before its penetration into skin 10 so that the cutting movement is carried out almost ballistically until the stop 36 is reached by the coupling member 30. For that, the transferred kinetic energy should be minimally about an order of magnitude above the cutting energy required for penetration in order to retain the very high speed during the forward movement into the tissue. A non-limiting example is as follows: for over 50% of the penetration depth d, the penetration speed can still be around about 15 m/sec.

After the fast penetration, the penetration element 14 is retracted in direction 43, as shown in FIG. 1c slowly together with the housing 32 by the second drive 40 in order to sample in the created pricking channel 46 blood and/or interstitial fluid via the collection slit 26. The retracting speed can amount to, for example, 0.1 m/sec, in which even an intermittent stoppage can be accommodated. It is appreciated that the retracting speed may vary, such that a retraction speed for the retraction in direction 41 of the penetration element 14 out of the skin 10 can be about 10% of the penetration speed or less. It is appreciated that in accordance with the present invention that the housing itself may act as a driver for the penetration element.

In FIGS. 1 to 6, different penetration devices are shown, each of which has a different actuating means for propelling a penetration movement of the penetration element to such a high penetration speed that a penetration depth variation caused by skin impression is less than about 0.3 mm. Each actuating means has a high-speed drive formed for the forward movement 41 of the penetration element 14 supported in the housing 32. Similar or same parts are provided with the same reference numbers as described above.

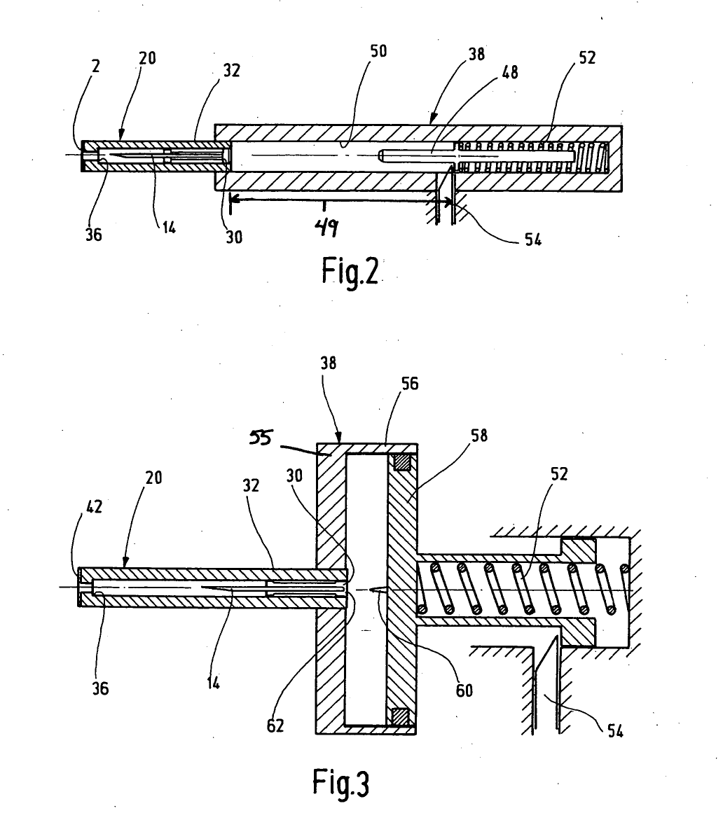

In the embodiment according to FIG. 2, a mechanical impulse is transferred from the actuator to the penetration element. The actuator has a driving body, which can be accelerated and decelerated or braked, separately from the penetration element, such as a plunger. A plunger 48 that is accelerated in a guide 50 over a comparatively long guide acceleration distance 49 by a preloaded helical compression spring 52 as soon as the latch 54 is removed. At the end of the guide acceleration distance 49 in guide 50, the plunger 48 transfers a high impulse to the penetration element 14 by striking on the coupling member 30, which in turn is stopped at the end of the penetration action by the stop 36 of the housing 32. A non-limiting example of a suitable preloaded or pre-compressed spring element is a silicone rubber spring, for the forward movement of the penetration element.

For the embodiment according to FIG. 3, a mechanical impulse is transferred from the actuator to the penetration element. The housing 32 is coupled to the bottom side 55 of the actuator, which is formed as a hydraulic or pneumatically operated cylinder 56 so as to be leak-proof. This cylinder 56 is designed with a short stroke and has a working volume that is larger as compared to the feed volume for the penetration element 14 of the housing 32. The piston 58 in the cylinder 56 is again driven by a spring 52 as soon as the latch 54 is removed. With this, a pressure is created, which accelerates the lightweight penetration element 14 over a relatively long distance at the desired speed. The working medium of the cylinder 56 can be a gas (for example air) or even a liquid (e.g. water, oil). An optional plug 60 on the piston 58 can serve to open a sealing foil 62 and to release the built up gas pressure on the penetration element 14. It is appreciated that it is within the scope of the present invention that the inner space of the housing 32 can be connected to a compensation volume before the penetration element 14 so that the counter-pressure does not slow down the penetration element.

FIG. 3 illustrates an embodiment of the invention wherein the spring 52 and the piston 58 have to be accelerated at very low speeds since the ratio of the surface area of the piston 58 to that of the coupling member 30 of the penetration element 14 causes a conversion to a very high speed. This makes a relatively small structure possible, especially if a flat disk spring is used as a moving spring instead of the helical spring 52 shown. Such disk springs have a strong regressive character, such that a maximum force is created at the end of the spring movement in the direction of the loading, to provide the maximum pressure as required in the cylinder 56.

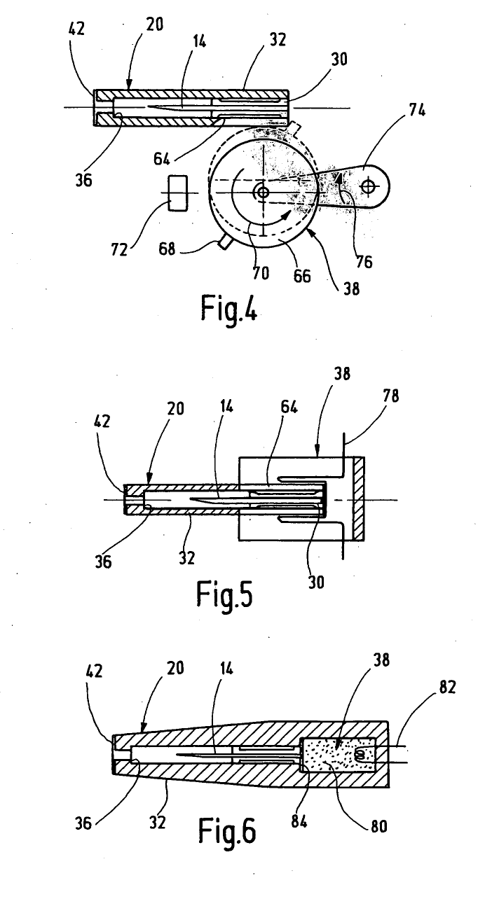

FIG. 4 shows an embodiment where a mechanical impulse is transferred from the actuator to the penetration element. The actuator has a driving body, which can be accelerated and decelerated or braked, separately from the penetration element, such as a flywheel. FIG. 4 illustrates that the penetration device includes an actuator having a flywheel drive 38. Here, there is a flywheel 66 beside the housing 32 provided with an engagement slot 64, which carries a radial projection 68. The flywheel 66 is set in the direction of the arrow 70 comparatively slowly, for example by an electric motor (not shown), until the peripheral speed of the projection 68 corresponds at least to the desired penetration element speed of at least about 15 m/sec. A sensor 72 (non-limiting examples of which include light barrier, hall sensor, reed contract, etc.) then creates a trigger signal, after which rocker 74 carrying the flywheel 66 is swung in the direction of the arrow 76 until the projection 68 comes in contact with the coupling member 30 of the penetration element 14. Penetration element 14 is in turn catapulted in with forward movement 29 toward the distal end 35 of the housing 32 like a punch until the stop 36 of the housing 32 stops the forward movement 29.

FIG. 5 shows an embodiment of the penetration device whereby the actuator is formed for the electrodynamic acceleration of the penetration element 14. In this case, an electromagnetic impulse triggers the forward movement of the penetration element. For example, an aluminium-foil tape 78 arranged meander-shaped is provided as high-speed drive for the penetration element 14. The aluminium tape 78 folds around the coupling member 30 of the penetration element 14 in the area of a slot opening 64 of the housing 32. For the triggering of the penetration movement, a current surge is fed through the aluminium tape 78, whereby it unfolds itself and accelerates the penetration element 14 with a very high-speed forward movement 29. This effect can be supported by applying a strong magnetic field in a suitable way through a magnetic device (not shown). The current surge can, a non-limiting example of which, occur through the discharging of condenser charged with about 100 Volt. For further electromagnetic force generation devices, reference is made to U.S. Pat. No. 5,928,192, issued Jul. 27, 1999, the disclosure of which is hereby incorporated by reference. In principle, such devices are also suitable for accelerating a lancing element 14 as payload.

FIG. 6 shows an embodiment of the penetration device whereby the actuator is formed by having a pyrotechnically operated form, wherein the penetration element 14 is brought at vary high speed by applying high pressure (about 100 to about 400 bar) over a comparatively short distance. As such, an excess pressure impulse triggers the forward movement of the penetration element. For this, a combustion chamber 79 is formed at the proximal end 37 of the housing 32, wherein a pyrotechnic propelling charge 80 is deposited, which may be ignited electrically by means of a heating wire 82. In order that the penetration element 14 does not move before the desired very high speed is reached, the combustion chamber 79 can be dammed with a film 84 of nitrocellulose. It is further contemplated that an indirect transfer of impulse be provided in accordance with the present invention. A non-limiting example of such an indirect transfer of impulse is through a plunger corresponding to the embodiment shown according to FIG. 2. In this case, the plunger is accelerated over a short distance and then moves the lightweight penetration element alone with itself.

As such, a penetration process and device for the extraction of body fluid is provided wherein a penetration element 14 is driven at a penetration speed for the penetration of the skin 10 of a body part 12 characterized in that the penetration speed of the penetration element 14 is selected to be so high that the penetration depth variation caused by skin impression is significantly reduced. In an embodiment, the penetration depth variation caused by skin impression is less than 0.3 mm. A penetration speed of more than 15 m/sec is used.

In a further embodiment, the penetration element 14 is driven in a non-controlled manner in the forward phase of the penetration movement. The penetration element 14 is propelled with a kinetic energy, which is at least an order of magnitude greater than the energy required for the penetration into the skin 10. Further, a surplus kinetic energy of the penetration element 14 at the end of the forward-directed penetration movement is transferred to the housing 32 as thrust. The body part 12 is pressed against an opening 22 of a portable instrument so that the skin 10 bulges inside the instrument in order to allow the penetration of the penetration element 14.

Having described the invention in detail and by reference to specific embodiments thereof, it will be apparent that modification and variations are possible without departing from the scope of the invention defined in the appended claims. More specifically, although some aspects of the present invention are identified herein, it is contemplated that the present invention is not necessarily limed to these aspects of the invention.

What is claimed is:

1. A method for extracting body fluid from a body part, the method comprising the steps of:
providing a penetration device including a penetration element formed to pierce the body part and an actuator formed to propel forward movement of the penetration element,
moving the penetration element toward the body part at a very high penetration speed to form an impression in the body part having a depth less than about 0.3 mm, and piercing the body part with the penetration element.

2. The method of claim 1 further comprising the step of pressing the body part against a pressing ring such that the body part bulges into the ring,

3. The method of claim 1 wherein the body part is a finger.

4. The method of claim 1 wherein the body fluid is blood.

5. The method of claim 1 wherein the penetration depth variation is less than about 0.2 mm.

6. The method of claim 1 wherein the speed of the penetration element when piercing the body part is at least about 15 m/sec.

7. The method of claim 1 wherein the speed of the penetration element when piercing the body part is about 20 m/sec to about 30 m/sec.

8. The method of claim 1 further comprising the step of retracting the penetration element from the body part at a speed of about 10% of the penetration speed or less.

9. The method of claim 1 wherein the moving step includes providing an electromagnetic impulse that triggers the forward movement of the penetration element.

10. The method of claim 1 wherein the moving step includes providing an excess pressure impulse that triggers the forward movement of the penetration element.

11. The method of claim 1 wherein the moving step includes transferring a mechanical impulse from the actuator to the penetration element.

12. The method of claim 1 wherein the moving step includes providing a preloaded spring element that triggers the forward movement of the penetration element.

13. The method of claim 1 wherein the moving step includes the step of actuating a flywheel.

14. The method of claim 1 wherein the moving step includes the step of actuating a plunger.

15. The method of claim 1 wherein the penetration device is positioned in a housing having a channel and the penetration element moves an acceleration distance in the channel.

16. The method of claim 15 wherein the housing is a driver for the penetration element during retracting movement of the penetration element from the body part.

17. The method of claim 1 wherein the penetration element is a lancet, a microsampler, or an integrated penetration and analyzing system.

18. A penetration device for the extraction of a body fluid, the device comprising:
a penetration element formed to pierce a skin of a body part, and actuating means for propelling a penetration movement of the penetration element, wherein the actuating means has a high-speed drive formed to bring the penetration element to such a high penetration speed that a penetration depth variation caused by skin impression is less than about 0.3 mm.

19. The device of claim 18 wherein the actuating means is formed to propel forward movement of the penetration element at a penetration speed of at least about 15 m/sec.

20. The device of claim 18 wherein the actuating means is formed to propel forward movement of the penetration element at a penetration speed of about 20 m/sec to about 30 m/sec.

21. The device of claim 18 wherein the device is formed for a retraction movement of the penetration element at a retracting speed of about 10% or less of the penetration speed.

22. The device of claim 18 wherein the penetration element has a shaft and a tip, the mass and tip together having a mass of about 25 mg or less.

23. The device of claim 18 wherein the actuating means provides an electromagnetic impulse.

24. The device of claim 18 wherein the actuating means has a first drive unit for the forward movement and a second drive unit for retracting movement of the penetration element.

25. The device of claim 18 wherein the actuating means provide an electromagnetic impulse for triggering the forward movement of the penetration element.

26. The device of claim 18 wherein the actuating means provides an excess pressure impulse.

27. The device of claim 26 wherein the actuating means is a pneumatic or hydraulic cylinder.

28. The device of claim 26 wherein the actuating means is a pyrotechnic propelling charge.

29. The device of claim 18 wherein the actuating means includes a preloaded spring element.

30. The device of claim 29 wherein the spring element is a precompressed silicone rubber spring.

31. The device of claim 18 wherein the actuating means is formed to transfer a mechanical impulse to the penetration element.

32. The device of claim 18 wherein the actuating means includes a driving body that can be accelerated and braked.

33. The device of claim 32 wherein the driving body is a flywheel or a plunger.

34. The device of claim 18 further comprising a housing having a channel and the penetration element is movable along an acceleration distance in the channel.

35. The device of claim 34 wherein the housing is the drive for the penetration element.

36. The device of claim 34 wherein the housing has proximal and distal ends and a stop positioned adjacent to the distal end.

37. The device of claim 18 wherein the penetration element is a lancet, a microsampler, or an integrated penetration and analyzing system.

38. A penetration kit for the extraction of body fluid from a body part, the kit comprising:
a pressing ring,
a penetration element sized for extension into the pressing ring and formed to pierce the body part,
a first actuator formed to propel forward movement of the penetration element at a penetration speed of at least about 15 m/sec, and
a second actuator formed to propel a retraction of the penetration element from the body part.

39. The kit of claim 38 wherein the first actuator is formed to propel forward movement of the penetration element at a penetration speed of about 20 m/sec to about 30 m/sec.

40. The kit of claim 38 wherein the first actuator provides an electromagnetic impulse.

41. The kit of claim 38 wherein the first actuator provides an excess pressure impulse.

42. The kit of claim 38 wherein the first actuator includes a preloaded spring element.

43. The kit of claim 38 wherein the first actuator is formed to transfer a mechanical impulse to the penetration element.

44. The kit of claim 38 further comprising a housing having a channel and the penetration element is movable along an acceleration distance in the channel.

45. The kit of claim 44 wherein the housing is the drive for the penetration element.

46. The kit of claim 44 wherein the housing has proximal and distal ends and a stop positioned adjacent to the distal end.

47. The kit of claim 38 wherein the penetration element is a lancet, a microsampler, or an integrated penetration and analyzing system.

48. The method of claim 38 wherein the penetration element is a lancet having a shaft and a tip, the mass and tip together having a mass of about 25 mg or less.

49. The kit of claim 38 wherein the second actuator formed to propel a retraction of the penetration element at a retracting speed of about 10% or less of the penetration speed.

50. The kit of claim 38 wherein the first actuator includes a plunger.

51. The kit of claim 38 wherein the first actuator includes a flywheel.
